(19) United States
(12) Patent Application Publication
(10) Pub. No.: US 2004/0047826 A1
(43) Pub. Date: Mar. 11, 2004

(54) Fragrance formulation containing pearlescent silicone oil beads

(75) Inventor: Richard Allen Brown, Northridge, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cosmetic Laboratories of America, a division of St. Ives Laboratories, Inc., Melrose Park, IL

(21) Appl. No.: 10/656,581

(22) Filed: Sep. 5, 2003

Related U.S. Application Data
(60) Provisional application No. 60/408,575, filed on Sep. 6, 2002.

(51) Int. Cl. A61K 7/06; A61K 7/11

(52) U.S. Cl. 424/70.12

(57) ABSTRACT

Disclosed is a composition comprising an aqueous phase; a silicone oil phase; and guanine, wherein the guanine is at the interface of the aqueous and silicone oil phases; and, optionally, one or more of the following ingredients: (i) pigment; (ii) dye; (iii) fragrance; (iv) emollient; (v) second silicone oil; (vi) glycol; (vii) vitamin; (viii) sun screen; (ix) sanitizing agent; and (x) acne treatment agent. Also disclosed is a spray assembly comprising (a) a container comprising a transparent wall and a pump assembly, which is optionally fluorinated, extending from the wall; (b) a liquid composition comprising a region containing pearlized oil droplets; and (c) a fluorinated dip tube extending from the pump assembly into the region of pearlized oil droplets of the composition. Further disclosed is a spray assembly comprising (a) a container comprising a transparent wall and a pump assembly extending from the wall; (b) the above composition; and (c) a dip tube extending from the pump assembly into a region comprising the guanine-containing silicone oil phase oil.

### CROSS-REFERENCE TO RELATED PATENT APPLICATIONS

This patent application claims the benefit of U.S. Provisional Patent Application No. 60/408,575, filed Sep. 6, 2002.

### FIELD OF THE INVENTION

This invention pertains to a composition having an aesthetically appealing appearance and to a spray assembly comprising a container and the composition.

### BACKGROUND OF THE INVENTION

Cosmetic products, such as those in the form of sprays, can have a variety of applications, such as, for example, a sanitizer (e.g., for hands or face), shine spray, perfume, air freshener, toner, moisturizer, body spray, colorant (e.g., for hair), sunscreen, and an acne treatment. Aside from the actual performance of the product for its intended performance, one important factor that drives consumers to select one cosmetic product over another is the visual appearance of the product, not only of the container, but also of the composition itself. A second important factor that distinguishes one cosmetic product over another, especially for spray-on products, is the feel of the product after it is applied to the skin.

There is a continuing effort to make many existing cosmetic products more effective by improving the “after-feel” of a product, for example, such that they do not render the skin greasy, and so that they have an improved visual appeal. Previous attempts to enhance the after-feel benefits and visual appeal of such products have not been fully satisfactory. For example, U.S. Pat. No. 6,270,782 (“the '782 patent”) discloses a composition comprising an oil phase, an aqueous phase, and a pigment in the form of particles, which are effective to form the oil phase into stable droplets. However, the oil phase of the composition is made up of mineral oil, which can leave a heavy or greasy feeling on the skin after application. In addition, according to the '782 patent, the container used to dispense the composition cannot contain a dip tube that extends into the droplet layer or else the droplet layer breaks, deforms, or clings to the container.

In addition, U.S. Pat. No. 3,920,883 (“the '883 patent”) discloses a cosmetic composition comprising oil, water, organic liquid miscible in water, and finely divided solid particles. The oil is dispersed in the form of spheres and the solid particles are substantially absorbed on the interface between the oil phase and the homogeneous mixture phase of the organic liquid and water. Many examples of oils and the solid particles are taught, but nothing in the '883 patent teaches preparing a composition with an improved after-feel.

Although cosmetic compositions containing pearlescent droplets have been previously known, such products have not been commercially feasible. Previous compositions have not been able to withstand the stresses of manufacturing and/or shipping. As a result, the cosmetic compositions deteriorate by, for example, the droplets coalescing, the pearlescent coating failing to adhere to the droplet surface, and/or the pearlescent coating becoming chalky looking.

Despite the availability of conventional aesthetically pleasing cosmetic compositions, it will be appreciated from the foregoing that there is a need in the art for a cosmetic composition with enhanced visual appeal and/or improved after-feel. Moreover, there is a need for such cosmetic compositions that are commercially feasible. It will be appreciated also that there is a need in the art for an assembly, in particular a spray assembly, which maintains the aesthetic appearance of the composition and allows for an effective delivery when the composition is applied to the skin.

### BRIEF SUMMARY OF THE INVENTION

The invention provides an aesthetically pleasing cosmetic composition with an improved after feel. In addition, the cosmetic composition is commercially viable in that the droplets remain stable, even during manufacture and shipping. Advantageously, the pearlescent coating remains intact on the oil droplets and does not slide off or become chalky in appearance. Thus, in one aspect, the invention provides a two-phase composition comprising an aqueous phase comprising an alcohol; a silicone oil phase; and guanine, wherein the guanine is at the interface of the aqueous and silicone oil phases.

The invention also provides a two-phase composition comprising an aqueous phase comprising an alcohol; a silicone oil phase; guanine, wherein the guanine is at the interface of the aqueous and silicone oil phases; and, optionally, one or more of the following ingredients: (i) pigment; (ii) dye; (iii) fragrance; (iv) emollient; (v) second silicone oil; (vi) glycol; (vii) vitamin; (viii) sun screen; (ix) sanitizing agent; and (x) acne treatment agent.

In another aspect, the present invention provides a spray assembly comprising (a) a container comprising a transparent wall and a pump assembly extending from the wall; (b) a liquid composition comprising a region containing pearlized oil droplets; and (c) a fluorinated dip tube extending from the pump assembly into the region of pearlized oil droplets of the composition. Optionally, the pump assembly is also fluorinated, particularly if the pump assembly is designed to come into contact with the composition.

In yet another aspect, the present invention provides a spray assembly comprising (a) a container comprising a transparent wall and a pump assembly extending from the wall; (b) a composition comprising an aqueous phase comprising an alcohol; a silicone oil phase; guanine, wherein the guanine is at the interface of the aqueous and silicone oil phases; and, optionally, one or more of the following ingredients: (i) pigment; (ii) dye; (iii) fragrance; (iv) emollient; (v) second silicone oil; (vi) glycol; (vii) vitamin; and (viii) sun screen; and (c) a dip tube extending from the pump assembly into a region comprising the silicone oil phase.

The present invention may be best understood with reference to the following detailed description of the preferred embodiments in conjunction with the accompanying drawings.

### BRIEF DESCRIPTION OF THE DRAWINGS

FIG. 1A illustrates a spherical spray assembly containing a dip tube that optionally comprises fluorinated polymeric material and a composition of the present invention.

FIG. 1B depicts a bottom view of the container shown in FIG. 1A.

FIG. 2A illustrates a conical spray assembly containing a dip tube that optionally comprises fluorinated polymeric material and a composition of the present invention.

FIG. 2B depicts a bottom view of the container shown in FIG. 2A.

### DETAILED DESCRIPTION OF THE INVENTION

The present invention provides a cosmetic composition that is aesthetically pleasing and has an improved after-feel when the composition is applied to the skin. The present invention is predicated, at least in part, on the surprising and unexpected discovery that a cosmetic composition comprising an aqueous phase, a silicone oil phase, and guanine, in which guanine resides at the interface of the two phases, exhibits improved skin feel and/or improved visual appeal. The improvement in these properties is not obtainable when the ingredients are used alone, and the improvement would not have been expected based on the properties of each ingredient, individually.

Accordingly, the present invention provides a two-phase composition comprising pearlescent silicone oil beads and water. The composition can be in the form of any of a wide variety of products for which such a two-phase composition is aesthetically appealing. By way of example, the beneficial effects of the invention are useful for cosmetic products such as, but not limited to, a sanitizer (e.g., for hands or face), shine spray, perfume, air freshener, toner, moisturizer, body spray, colorant (e.g., for hair), sunscreen, acne treatment, and the like. In preferred embodiments, the composition is suitable for use as a spray.

The two-phase composition in accordance with the invention comprises an aqueous phase and a silicone oil phase, as well as guanine, which resides at the interface of the aqueous and silicone oil phases. In addition, the composition preferably comprises an alcohol. Visually, when packaged, the composition is designed so that the silicone oil phase is in the form of droplets (or beads) in a lower region of the packaging. The aqueous phase resides in the interstices between the droplets and in a separate region generally above the silicone oil droplets. In order to achieve suitable relative densities between the two phases, the alcohol is added to the composition so as to achieve the desired arrangement of the silicone oil phase in the lower region of the container and the water phase in the interstices and above the silicone oil phase. In some embodiments, the alcohol is in the silicone oil phase as well as the water phase. Preferably, the silicone oil droplets are provided such that they are only present at most in the lower half of the packaging, as discussed in more detail herein below. It is to be noted that, although the singular is utilized for convenience to describe certain ingredients, it will be appreciated that the use of the singular herein is meant to encompass one or more of the recited ingredients, unless otherwise indicated.

In accordance with the present invention, the guanine imparts a pearlized effect on the silicone oil droplets so that the droplets look visually appealing (e.g., sparkling). Significantly, the guanine also provides the surprising and unexpected result of preventing the silicone oil droplets from coalescing excessively. Other pearlizing compounds, such as mica and other powdered materials, do not provide a stable composition in conjunction with the silicone oil. Particularly, the guanine prevents the silicone oil droplets from exceeding a certain size, as desired. While not wishing to be bound by any particular theory, it is believed that the guanine coats the silicone oil droplets and acts at the interface of the oil droplets and water to prevent the silicone oil droplets from agglomerating beyond a certain point so as to avoid the formation of excessively large or indistinguishable silicone oil droplets. Preferably, the guanine is derived from a natural source (e.g., fish scales) rather than prepared synthetically. In general, naturally derived guanine typically provides an enhanced pearlescent effect compared to synthetic guanine. It is to be noted that fish scale powder is not necessarily the same as guanine, as fish scales contain a myriad of other components, which could affect the desired composition. Optionally, fish scales are enzymatically treated in order to provide guanine suitable for the present invention.

The guanine can be present in any effective amount so that the size of the silicone oil droplets is controlled, as desired. For example, preferably the silicone oil droplets have a mean diameter of from about 1 mm to about 10 mm (e.g., a mean diameter of from about 2 mm to about 5 mm). Thus, in order to control the size of the silicone oil droplets, as desired, in some embodiments, the guanine is present in an amount of from about 0.02% to about 0.3%, on a dry basis, by weight of the composition. In this respect, in some embodiments, the guanine is provided in the form of a slurry. For example, the slurry can contain solid particles of guanine with other ingredients, as desired, such as, for example, surfactants (e.g., polysorbate 80 or the like), water, and an alcohol. One example of a suitable guanine-containing slurry is Mearlmaid Okla., available from Englehard Corporation, which contains about 24% guanine by weight. By “dry basis,” it is meant that the amount of guanine refers only to guanine and excludes the other ingredients in the guanine slurry.

In some embodiments, the guanine is silanized (treated with silicone) such that a chemical bond is formed between Si and guanine. By way of example, the guanine can be treated with a silane such as, but not limited to, dimethyl siloxane or trimethyl siloxane, as will be readily appreciated by one of ordinary skill in the art.

Any suitable silicone oil can be included in the composition. By way of example, the silicone oil can be selected from trisiloxane, cyclomethicone, dimethicone, phenyltrimethicone, and the like, or combinations thereof. The silicone oil can be present in any suitable amount. For example, the silicone oil can be present in an amount of from about 5% to about 50% by weight of the composition, more preferably from about 20% to about 30% by weight of the composition.

Preferably, upon addition of the pearlizing agent, the silicone oil phase forms at least about 20 droplets and more preferably forms at least about fifty droplets. The pearlized oil droplets preferably are present in an amount of from about 20% to about 50% by volume (more preferably about 25% to about 40%) of the composition.

The water can be present in any suitable amount. For example, the water can be present in an amount of from about I 0% to about 25% by weight of the composition, more preferably from about 14% to about 18% by weight of the composition.

Any suitable alcohol can be used. For example, the alcohol can be selected from ethanol, isopropyl alcohol, and the like, or combinations thereof. The alcohol can be present in any suitable amount so that it provides the desired density balance between the aqueous and silicone oil phases so that the composition is arranged with the aqueous phase situated generally above the silicone oil phase in the packaging. For example, depending on the constituents of the aqueous and silicone oil phases, in some embodiments the alcohol can be present in an amount of from about 35% to about 75% by weight of the composition, more preferably from about 40% to about 50% by weight of the composition, even more preferably, about 45% by weight of the composition. Advantageously, the presence of the alcohol minimizes the ability of microorganisms to grow and typically obviates the need for any additional preservative to be added to the composition. If necessary or desired, additional known preservatives can be added to the composition.

The composition optionally can further comprise any suitable glycol additive. Typical glycol additives include propylene glycol, dipropylene glycol, 2-methyl-1,3-propanediol (MPdiol), butylene glycol, dibutylene glycol, pentylene glycol, hexylene glycol, and the like, and combinations thereof. If included, the glycol is present in any suitable amount, such as from about 0.01% to about 2% by weight of the composition. In some preferred embodiments, glycol is not added.

The composition optionally can further comprise a fragrance, if desired. The fragrance can be any fragrance suitable for cosmetic preparations, but preferably, the fragrance is substantially free of surfactant. A fragrance that is substantially free of surfactant is one in which upon addition to the composition, it does not cause an emulsion to form. Preferably, the fragrance comprises less than about 1% by weight of surfactant, more preferably less than about 0.5% by weight of surfactant, and most preferably less than about 0.1% by weight of surfactant. Suitable fragrances include, but are not limited to, well known fragrances such as those commercially available, as will be readily appreciated by the ordinarily skilled artisan. If included, the fragrance is present in an amount of from about 0.1% to about 10% by weight of the composition.

The composition optionally comprises an emollient, such as, for example, a silicone oil, a vegetable oil, hydrocarbon oils that are preferably light and non-greasy (e.g., having from about 6 to about 20 carbon atoms) such as petrolatum, mineral oil, isostearyl alcohol, and combinations thereof. If present, the emollient is present in an amount of from about 0.1% to about 20% by weight of the composition. Since silicone oil present in the silicone phase of the composition already acts as an emollient, in some preferred compositions, an additional emollient is not necessary. In other preferred compositions, such as hair products used to impart shine, an additional emollient can be desirable.

The composition can further comprise a pigment. The pigment can be in the form of any pigment known to be suitable for cosmetic preparations. Preferably, the pigment is one that does not cause the pearlescent beads to coalesce in the two-phase composition. The pigment preferably is selected from the group consisting of titanium dioxide, chromium hydroxide, yellow iron oxide, red iron oxide, black iron oxide, zinc oxide, talc, mica, magnesium carbonate, calcium carbonate, magnesium silicate, aluminum magnesium silicate, silica, ultramarine, nylon powder, polyethylene powder, polystyrene powder, silk powder, crystalline cellulose, starch, titanated mica, iron oxide titanated mica, bismuth oxychloride, boron nitride, and combinations thereof. If present, the pigment is present in an amount of from about 0.001% to about 0.1% by weight of the composition. In some preferred compositions, a pigment is not added.

The composition optionally comprises a dye such as approved FD&C dyes, approved D&C dyes, and approved external D&C dyes, which will be readily known to those skilled in the art. Suitable dyes include, for example, Blue Nos. 1 and 4, Brown No. 1, Green Nos. 3, 5, 6, and 8, Orange Nos. 4, 5, 10, and 11, Red Nos. 4, 6, 7, 17, 21, 22, 27, 28, 30, 31, 33, 34, 36, and 40, Violet No. 2, Yellow Nos. 2, 5, 6, 7, 8, 10, and 11. If included, the dye is present in an amount of from about 0.000001% to about 0.005% by weight of the composition. Preferably the dye is present in an amount of from about 0.00001% to about 0.00008% by weight of the composition.

The composition optionally can further comprise a second silicone oil to facilitate imparting shine. The second silicone oil is selected from the group consisting of phenyl trimethicone, cetyl methicone, isostearyl methicone, and combinations thereof. If present, the second silicone oil can be present in any suitable amount, such as an amount of from about 0.1 % to about 30% by weight of the composition.

The composition optionally can further comprise one or more vitamins, if desired. The vitamin can be any vitamin suitable for cosmetic applications. Preferably, however, the vitamin is oil soluble. The vitamin preferably is selected from the group consisting of Vitamin E, Vitamin K, Vitamin C and derivatives thereof, Vitamin A, B Vitamins, and combinations thereof. If included, the vitamin is present in an amount of from about 0.001% to about 3% by weight of the composition. The amount of vitamin(s) used depends on the specific vitamin desired. For example, in some preferred compositions, the composition comprises Vitamin E, preferably in an amount of from about 0.1 % to about 1% by weight of the composition.

The water added to the composition can have any suitable pH. Since the composition is to be applied to the hair or skin, typically the water added to the composition has a pH of from about 2 to about 9. Preferably, the water added to the composition has a pH of from about 5 to about 7.

The composition of the present invention is useful for the preparation of cosmetic products such as, but not limited to, a sanitizer (e.g., for hands or face), shine spray, perfume, air freshener, toner, moisturizer, body spray, colorant (e.g., for hair), sunscreen, and acne treatment. As will be appreciated by one of ordinary skill in the art, depending on the desired use of the product, the composition can be varied accordingly without departing from the aesthetic quality of the two-phase composition comprising pearlescent droplets. For example, if the composition is to be used for a body spray, perfume, or air freshener, the amount of fragrance present in the composition can be increased. If a toner is desired, the composition can contain a higher amount of alcohol. For acne treatment products, the addition of salicylic acid, colloidal sulfur, benzoyl peroxide, triclosan, and other suitable acne treatment agents (as will be readily appreciated by an ordinarily skilled artisan) can be desirable. Similarly, triclosan or other similar antibacterial agents, as will be appreciated by one of ordinary skill in the art, can be added to compositions for use as a sanitizer.

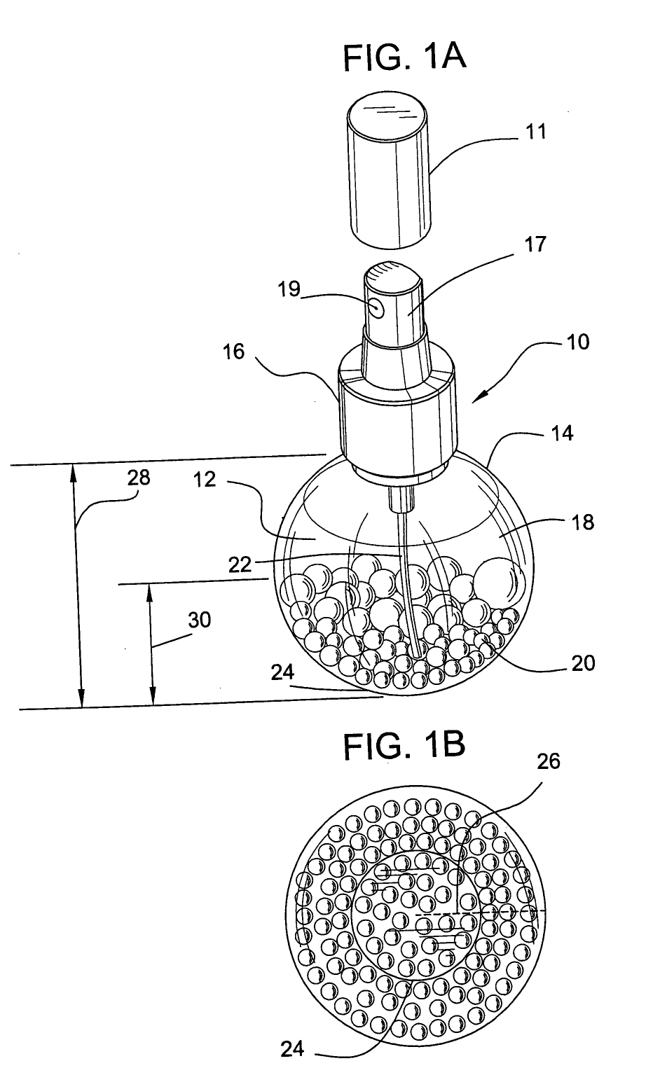

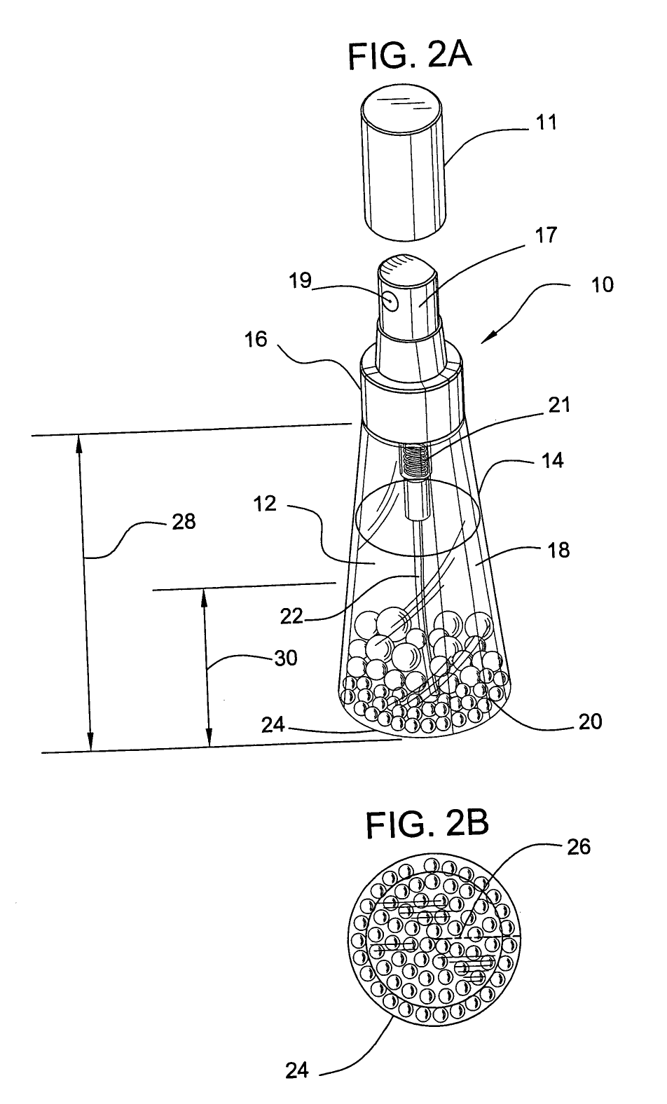

Referring now to the figures, in another aspect of the present invention, a spray assembly 10 is provided. The spray assembly 10 comprises a container 12 comprising a transparent wall 14 and a pump assembly 16 extending from the wall 14 and a cap 11 for the container. As one of ordinary skill in the art will appreciate, as seen in FIGS. 1A and 2A, the pump assembly 16 typically comprises an actuator 17 that is movable up and down and a spray nozzle 19 that is attached to the actuator 17. As shown in FIG. 2A, a spring 21 is compressed when the actuator 17 is depressed and provides restoring force when released. Contained within the spray assembly 10 is a liquid composition 18, which comprises a region containing pearlized oil droplets 20. In some embodiments, as seen, for example, in FIGS. 1A and 2A, a dip tube 22 extends from the pump assembly 16 into the region of pearlized droplets 20.

It will be appreciated that the container 12 of the spray assembly 10 can be any suitable shape. As seen in the FIGS., however, preferably the wall 14 is spherical, bulbous, or conical. Furthermore, the spray assembly 10 can be in the form of a shaker device, in which the liquid composition is shaken or poured from one or more openings in the assembly. In such embodiments, a dip tube is not required.

The transparent wall 14 preferably is formed, at least in part, of glass or a polymeric material. The polymeric material for the transparent wall 14 preferably is selected from the group consisting of one or more polyethylene terephthalate (PET), polyvinylchloride (PVC), co-polymers thereof, and blends thereof. The dip tube 22 and/or pump assembly 16 preferably is formed, at least in part, from a polymeric material. The polymeric material for the pump assembly 16 and/or the dip tube 22 preferably is selected from the group consisting of polyethylene (e.g., LDPE, HDPE, and mixtures thereof), polypropylene (e.g., isotactic, syndiotactic, atactic, and mixtures thereof), co-polymers thereof, and blends thereof. In preferred embodiments, the polymeric material for the pump assembly 16 and/or the dip tube 22 is fluorinated. In an especially preferred embodiment, the pump assembly 16 and/or the dip tube 22 comprises fluorinated polyethylene.

The container 12 preferably further comprises a base 24 and a radius 26 at the largest diameter of the container 12 that is from about 1 cm to about 4 cm (see FIGS. 1B and 2B). The container 12 preferably is such that the transparent wall 14 defines a height 28, and half of the volume of the spray assembly is equal to or less than about half of the wall height 28. In other words, the container 12 preferably has a shape that enables the majority (preferably all) of the silicone phase of the composition 18 to reside in the lower half 30 of the container 12. Preferably, the container is at least 1 oz in size, and more preferably the container is about 1 oz to about 8 oz in size. To accentuate the aesthetic property of the droplets, the shape of the transparent wall 14 can be constructed so as to magnify the droplets, if desired. As seen in FIG. 1A, the wall 14 is preferably curved, which changes the refractive index thereby producing a lens effect and magnifying the size appearance of certain of the droplets in the composition, which is desirably transparent so that the visual benefits are realized.

The following example further illustrates the invention but, of course, should not be construed as in any way limiting its scope.

### EXAMPLE

This example describes the preparation of compositions of the present invention.

The ingredients and amounts listed in Tables 1-4 were combined using the following method to prepare the compositions. The alcohol was added to a flask at room temperature. The guanine, any glycols, any dyes, any fragrance, and silicone oil(s) were added to the alcohol. If one or more pigments were used, they were first added to the silicone oil(s) and added along with the oil(s). The mixture was stirred with a paddle mixer to avoid incorporating large amounts of air. After breaking up any large chunks, water was added. As the mixture was agitated, it was transferred to a hopper and used to fill containers. Once agitation was ceased, the two phases separated, and the pearlescent droplets formed within the aqueous solution.

TABLE 1
Perfume Spray A

| | | | Amount | Weight |
|---|---|---|---|---|
| Ingredient | Supplier | Description | (g) | % |
| SD Alcohol | Remet Corp. | SD Alcohol 40-B | 395.28 | 49.41 |
| 40-B 200 | | | | |
| Butylene | | | 6.80 | 0.85 |
| Glycol | | | | |
| Mearlmaid OL | Engelhard | Isopropyl Alcohol, | 0.88 | 0.11 |
| B26562 | | Guanine, and | | |
| | | Polysorbate 80 | | |
| Red #33 | Warner | Dye | 0.24 | 0.03 |
| (0.3%) | Jenkinson | | | |
| Red #28 | Warner | Dye | 0.24 | 0.03 |
| (0.03%) | Jenkinson | | | |
| DC 345 | Dow Corning | Cyclomethicone | 14.00 | 1.75 |
| DC 2-1184 | Dow Corning | Trisiloxane and | 219.12 | 27.39 |
| | | Dimethicone | | |
| Fragrance | Firmenich | Fragrance | 24.00 | 3.00 |
| Deionized | | | 139.44 | 17.43 |
| Water | | | | |
| TOTAL | | | 800.000 | |

TABLE 2
Perfume Spray B

| | | | Amount | Weight |
|---|---|---|---|---|
| Ingredient | Supplier | Description | (g) | % |
| SD Alcohol | Remet Corp. | SD Alcohol 40-B | 395.632 | 49.45 |
| 40-B 200 | | | | |
| Butylene | | | 6.800 | 0.85 |
| Glycol | | | | |
| Mearlmaid OL | Engelhard | Isopropyl Alcohol, | 0.640 | 0.08 |
| EP021230 | | Guanine, and | | |
| | | Polysorbate 80 | | |
| RAB 020312C | Cardre and | Chromium | 0.064 | 0.008 |
| | Dow Corning | Hydroxide Green, | | |
| | | Methicone, Iron | | |
| | | Oxide, Trisiloxane | | |
| | | and Dimethicone | | |
| DC 345 | Dow Corning | Cyclomethicone | 14.032 | 1.754 |
| DC 2-1184 | Dow Corning | Trisiloxane and | 219.256 | 27.407 |
| | | Dimethicone | | |
| Fragrance | Firmenich | Fragrance | 24.040 | 3.005 |
| Deionized | | | 139.520 | 17.44 |
| Water | | | | |
| TOTAL | | | 799.984 | |

TABLE 3
Toner A

| | | | Amount | Weight |
|---|---|---|---|---|
| Ingredient | Supplier | Description | (g) | % |
| SD Alcohol | Remet Corp. | SD Alcohol 40-B | 934.0 | 46.70 |
| 40-B 200 | | | | |
| DC 2-1184 | Dow Corning | Trisoloxane and | 758.2 | 37.91 |
| | | Dimethicone | | |
| Fragrance | Shaw Mudge | Fragrance | 12.6 | 0.63 |
| Violet 2 | Warner | Dye | 1.6 | 0.08 |
| (0.05%) | Jenkinson | | | |
| Mearlmaid OL | Engelhard | Isopropyl Alcohol, | 3.0 | 0.15 |
| | | Guanine, and | | |
| | | Polysorbate 80 | | |
| Deionized | | | 290.6 | 14.53 |
| Water | | | | |
| TOTAL | | | 2000.0 | |

TABLE 4
Shine Spray A

| | | | Amount | Weight |
|---|---|---|---|---|
| Ingredient | Supplier | Description | (g) | % |
| SD Alcohol | Remet Corp. | SD Alcohol 40-B | 547.320 | 45.61 |
| 40-B 200 | | | | |
| Butylene | | | 5.880 | 0.490 |
| Glycol | | | | |
| Red 33 | Warner | Dye | 0.240 | 0.020 |
| (0.3%) | Jenkinson | | | |
| Mearlmaid | Engelhard | Isopropyl Alcohol, | 1.800 | 0.150 |
| OL | | Guanine, and | | |
| | | Polysorbate 80 | | |
| Deionized | | | 209.832 | 17.486 |
| Water | | | | |
| Silicone 556 | Dow | Phenyl Trimethicone | 18.000 | 1.500 |
| | Corning | | | |
| DC 345 | Dow | Cyclomethicone | 24.000 | 2.000 |
| | Corning | | | |
| SD Alcohol | Remet Corp. | SD Alcohol 40-B | 48.000 | 4.000 |
| 40-B 200 | | | | |
| DC 2-1184 | Dow | Trisiloxane and | 338.880 | 28.240 |
| | Corning | Dimethicone | | |
| Fragrance | Shaw Mudge | Fragrance | 6.000 | 0.500 |
| Chromium | | | 0.048 | 0.004 |
| HYD SI AS | | | | |
| TOTAL | | | 1200.000 | |

### COMPARATIVE EXAMPLE

This example describes the preparation of a perfume spray containing pearlizing agents other than guanine.

The ingredients and amounts listed in Table 5 were combined using the method described in the previous example. Various powdered pigments and pearlizing agents were added to the two-phase composition in comparison to guanine (entries 2-4). The number of droplets and their appearance was observed 2 h after addition of the pearlizing agent. The same observation was made again 4 days after the addition, unless the initial results were unsatisfactory. The results and observations are summarized in Table 6. The terms “½ coat,” “¾ coat,” etc. signify that only the bottom half or bottom three-quarters of the droplets (and so on) appeared colored-coated or pearly. In other words, the pigment did not adhere well to the droplet. The term “color coat” means that the droplets were colored but not pearlescent.

TABLE 5
Perfume Spray C

| | | | Weight |
|---|---|---|---|
| Ingredient | Supplier | Description | % |
| SD Alcohol 40-B 200 | Remet Corp. | SD Alcohol 40-B | 55.00 |
| Butylene Glycol | | | 0.48 |
| Mint Green (0.3%) | | Dye | 0.03 |
| Blue #1 (0.03%) | | Dye | 0.03 |
| DC 2-1184 | Dow Corning | Trisiloxane and | 25.00 |
| | | Dimethicone | |
| Fragrance | | Fragrance | 3.00 |
| Deionized Water | | | 16.37 |
| TOTAL | | | 99.91 |

TABLE 6

| | | | | | # of | Droplet | # of | Droplet |
|---|---|---|---|---|---|---|---|---|
| | | | | Weight | droplets | appearance | droplets | appearance |
| Entry | Tradename | Ingredient | Supplier | % | after 2 h | after 2 h | after 4 d | after 4 d |
| 1 | Blank | — | — | — | 1 | No coat | | |
| 2 | Mearlmaid OL | Guanine | Engelhard | 0.083 | 5 | Coated | 5 | Poor |
| 3 | Mearlmaid OL | Guanine | Engelhard | 0.127 | >50 | Uniform | >50 | Best |
| 4 | Mearlmaid OL | Siliconized | | 0.097 | >50 | Uniform | >50 | Poor |
| | siliconized in | guanine | | | | | | |
| | acetone 34.4% | | | | | | | |
| 5 | Mearlmica- | Mica, lauroyl | Engelhard | 0.042 | >20 | Coated | >20 | Poor |
| | treated SVA | lysine | | | | | | |
| 6 | Timiron MP- | Mica, TiO₂, | Rona | 0.042 | 1 | ½ coat | | |
| | 111 Ultraluster | silica | | | | | | |
| | Cosmetic | | | | | | | |
| | Pigment | | | | | | | |
| 7 | Timiron MP- | Mica, TiO₂, | Rona | 0.055 | 1 | No coat | | |
| | 1001 | silica | | | | | | |
| | Supersheen | | | | | | | |
| | Cosmetic | | | | | | | |
| | Pigment | | | | | | | |
| 8 | Platinum Super | Aluminum | Presperse | 0.054 | 3 | ¾ coat | 1 | |
| | | Powder | | | | | | |
| 9 | Timiron | Mica, TiO₂, | Cardre | 0.045 | 1 | Pearl coat | — | Color coat |
| | Supersheen MP | silica | | | | | | |
| | 1001 AS | | | | | | | |
| 10 | Chromium | Chromium | Sun | 0.050 | 1 | No coat | | |
| | Oxide Green SI | Oxide | Chemical | | | | | |
| 11 | Chromium | Chromium | Cardre | 0.650 | 1 | Green on | | |
| | Oxide Green SI | Oxide | | | | bottom | | |
| 12 | 40% 70257 | TiO₂ in | Cardre | 0.7801 | Rough | | | |
| | U.F. Titanium | octyldodecyl | | | coat | | | |
| | Dioxide AS in | neopentanoate | | | | | | |
| | Elefac I-205 | | | | | | | |
| 13 | Pearl Copper | Mica, TiO₂ | Cardre | 0.042 | 1 | Copper coat | ⅘ coat | |
| | 1000 SI2 | | | | | | | |
| 14 | Flamenco Red | Pigment | Cardre | 0.054 | 1 | Red pearl | — | Color coat |
| | AS | | | | | coat | | |
| 15 | 50% Yellow | Iron Oxide | Cardre | 0.049 | 1 | Yellow coat | — | Color coat |
| | Iron | | | | | | | |
| | Oxide/PMMA | | | | | | | |
| | AS | | | | | | | |
| 16 | Yellow Iron | Iron Oxide | Cardre | 0.040 | 2 | ½ yellow | 1 | |
| | Oxide (1010) | | | | | coat | | |
| | LTM | | | | | | | |
| 17 | 3M Cosmetic | Magnesium | Cardre | 0.061 | 2 | ½ coat | 1 | |
| | Microspheres | silicate | | | | | | |
| | CM-111 | | | | | | | |
| 18 | Black Iron | Iron Oxide | Cardre | 0.033 | 3 | ½ coat | 1 | |
| | Oxide (1080) | | | | | | | |
| | LTM | | | | | | | |
| 19 | 50% | Pigment | Cardre | 0.067 | >10 | Blue coat | >10 | Ok |
| | ultramarine | | | | | | | |
| | Blue/PMMA | | | | | | | |
| | AS | | | | | | | |
| 20 | Timiron Super | Mica, TiO₂ | Rona | 0.041 | 3 | ½ coat | 1 | |
| | Silver cosmetic | | | | | | | |
| | pigment | | | | | | | |
| 21 | 3M Cosmetic | Magnesium | Cardre | 0.130 | 2 | Non- | 2 | |
| | Microspheres | silicate | | | | uniform | | |
| | CM-111 PP | | | | | | | |
| 22 | Duocrome | Mica, TiO₂, | Engelhard | 0.175 | 1 | No coat | | |
| | DBV | ferric | | | | | | |
| | | ferrocyanide | | | | | | |
| 23 | Titanium | TiO₂ | Cardre | 0.045 | 1 | White coat | | Color coat |
| | Dioxide SI2 | | | | | | | |
| 24 | Biron Fines | Bismuth | Rona | 0.075 | >20 | Pearl coat | >20 | Ok |
| | Cosmetic | oxychloride | | | | | | |
| | Pigment | | | | | | | |
| 25 | Red Iron Oxide | Ferric Oxide | Cardre | 0.040 | 1 | Red coat | | Color coat |
| | AS | | | | | | | |
| 26 | Yellow Iron | Iron Oxide | Cardre | 0.081 | 1 | Yellow coat | | Color coat |
| | Oxide AS | | | | | | | |
| 27 | Red Iron Oxide | Ferric Oxide | Cardre | 0.046 | 1 | Red coat | | Color coat |
| | SI2 | | | | | | | |
| 28 | BTD-11S2 | TiO₂, isopropyl | Kobo | 0.077 | 1 | White coat | | Color coat |
| | | titanium | | | | | | |
| | | triisostearate | | | | | | |
| 29 | Luxsil | Calcium | Argan | 0.098 | 1 | ½ coat | | |
| | | aluminum | | | | | | |
| | | borosilicate | | | | | | |
| 30 | Sericite AS | Sericite | Cardre | 0.074 | 1 | Non- | | |
| | | | | | | uniform | | |
| 31 | Mica 8 AS | Mica | Cardre | 0.063 | 1 | Non- | | |
| | | | | | | uniform | | |
| 32 | Dichrona BG | Mica, TiO₂, | Rona | 0.063 | 1 | No coat | | |
| | Cosmetic | ferric | | | | | | |
| | Pigment | ferricyanide | | | | | | |
| 33 | PF-5 TiO₂ Cr- | TiO₂, | Kobo | 0.054 | 1 | White coat | | Color coat |
| | 50 | perfluoroalkyl | | | | | | |
| | | phosphate | | | | | | |
| 34 | Sicopearl | Iron oxide, | BASF | 0.057 | 1 | ½ coat | | |
| | Fantastico | silica | | | | | | |
| | Ruby | | | | | | | |
| 35 | BBO-11S2 | Iron oxide, | Kobo | 0.060 | 1 | ½ coat | | |
| | | isopropyl | | | | | | |
| | | titaniuim | | | | | | |
| | | triisostearate | | | | | | |
| 36 | Timiron Super | Mica, TiO₂, | Cardre | 0.063 | 1 | Pearl coat | | Color coat |
| | Silver Fine AS | silica | | | | | | |
| 37 | Cloisonne Gold | Mica, TiO₂, | Engelhard | 0.028 | 1 | ¼ coat | | Color coat |
| | 222C | iron oxide | | | | | | |
| 38 | Chroma-lite | Mica, bismuth | Engelhard | 0.022 | 2 | Black coat | 2 | Color coat |
| | black | oxychloride, | | | | | | |
| | | iron oxide | | | | | | |
| 39 | Extender W | Mica, TiO₂, | Rona | 0.055 | 1 | ½ coat | | |
| | | talc | | | | | | |
| 40 | Microna Matte | Mica, ferric | Rona | 0.039 | 1 | No coat | | |
| | Blue Cosmetic | ferrocyanide | | | | | | |
| | Pigment | | | | | | | |
| 41 | Timiron MP-17 | Mica, TiO₂, | Rona | 0.040 | 1 | Non- | | |
| | Satin | silica | | | | uniform | | |
| 42 | Timiron MP- | Mica, TiO₂, | Rona | 0.031 | 2 | ¼ coat | | |
| | 1117 Satin | silica | | | | | | |
| | Cosmetic | | | | | | | |
| | Pigment | | | | | | | |
| 43 | Micronasphere | Mica, silica | Rona | 0.029 | 1 | Non- | | |
| | M | | | | | uniform | | |
| 44 | Graphite Extra | Graphite | | 0.021 | 5 | Spotty coat | | |
| | Fine | | | | | | | |
| 45 | Chromium | Chromium | Cardre | 0.035 | >50 | Green coat | >50 | Ok |
| | Oxide Green | Oxide | | | | | | |
| | AS | | | | | | | |
| 46 | Chromium | Chromium | Cardre | 0.085 | 1 | Green coat | | Color coat |
| | Hydroxide | Hydroxide | | | | | | |
| | Green Si | | | | | | | |
| 47 | Teflon | Polytetrafluoro- | | 0.062 | 1 | No droplets | | |
| | | ethylene | | | | | | |
| 48 | Flamenco | Mica, TiO₂ | Cardre | 0.048 | 1 | Non- | | |
| | Velvet | | | | | uniform | | |
| | 120VAS | | | | | | | |
| 49 | Mearlmica | Mica, lauroyl | Cardre | 0.085 | 1 | Pearl coat | | Color coat |
| | SVAS | lysine | | | | | | |

As seen in Table 6, the majority of powdered pigments and pearlizing agents caused the silicone oil phase to coalesce into only one or two droplets. Moreover, several of the additives did not provide a uniform colored/pearlescent coating on the oil droplets. Chrominium oxide green AS (entry 46) allowed adequate formation of the oil droplets with a uniform green coating. Use of this pigment, however, is not desirable due to its lack of pearlescence and aesthetically pleasing appearance for the purposes of the invention. It is noteworthy that chromium oxide green and other suitable pigments (see Table 6) can be used, however, in combination with guanine in order to provide a colored pearlescent droplet.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A two-phase composition comprising:
(a) an aqueous phase comprising an alcohol;
(b) a silicone oil phase; and
(c) guanine, wherein the guanine is present at the interface of (a) and (b).

2. The composition of claim 1, wherein the silicone oil phase is in the form of droplets.

3. The composition of claim 2, wherein the droplets have a mean diameter of from about 1 mm to about 10 mm.

4. The composition of claim 3, wherein the droplets have a mean diameter of from about 2 mm to about 5 mm.

5. The composition of claim 1, wherein water is present in an amount of from about 10% to about 25% by weight of the composition.

6. The composition of claim 1, wherein the silicone oil is selected from the group consisting of trisiloxane, cyclomethicone, dimethicone, phenyltrimethicone, and combinations thereof.

7. The composition of claim 1, wherein the silicone oil is present in an amount of from about 5% to about 50% by weight of the composition.

8. The composition of claim 1, wherein the guanine is silanized with a silicone oil.

9. The composition of claim 1, wherein the guanine is present in an amount of from about 0.02% to about 0.3% by weight of the composition.

10. The composition of claim 1, wherein the alcohol is selected from the group consisting of ethanol, isopropyl alcohol, and combinations thereof.

11. The composition of claim 1, wherein the alcohol is present in an amount of from about 35% to about 75% by weight of the composition.

12. The composition of claim 1, further comprising a glycol.

13. The composition of claim 12, wherein the glycol is selected from the group consisting of propylene glycol, dipropylene glycol, 2-methyl-1,3-propanediol (MPdiol), butylene glycol, dibutylene glycol, pentylene glycol, hexylene glycol, and combinations thereof.

14. The composition of claim 12, wherein the glycol is present in an amount of from about 0.01% to about 2% by weight of the composition.

15. The composition of claim 1, further comprising a fragrance.

16. The composition of claim 15, wherein the fragrance is substantially free from surfactant.

17. The composition of claim 15, wherein the fragrance is present in an amount of from about 0.1% to about 10% by weight of the composition.

18. The composition of claim 1, further comprising an emollient.

19. The composition of claim 18, wherein the emollient is selected from the group consisting of a silicone oil, a vegetable oil, petrolatum, mineral oil, isostearyl alcohol, hydrocarbon oil having from about 6 to about 20 carbon atoms, and combinations thereof.

20. The composition of claim 18, wherein the emollient is present in an amount of from about 0. 1% to about 20% by weight of the composition.

21. The composition of claim 1, further comprising a pigment.

22. The composition of claim 21, wherein the pigment is selected from the group consisting of titanium dioxide, chromium hydroxide, yellow iron oxide, red iron oxide, black iron oxide, zinc oxide, talc, mica, magnesium carbonate, calcium carbonate, magnesium silicate, aluminum magnesium silicate, silica, ultramarine, nylon powder, polyethylene powder, polystyrene powder, silk powder, crystalline cellulose, starch, titanated mica, iron oxide titanated mica, bismuth oxychloride, boron nitride, and combinations thereof.

23. The composition of claim 21, wherein the pigment is present in an amount of from about 0.001% to about 0.1% by weight of the composition.

24. The composition of claim 1, further comprising a dye.

25. The composition of claim 24, wherein the dye is selected from the group consisting of FD&C dyes, D&C dyes, external D&C dyes, and combinations thereof.

26. The composition of claim 24, wherein the dye is present in an amount of from about 0.000001% to about 0.005% by weight of the composition.

27. The composition of claim 1, further comprising a second silicone oil to facilitate imparting shine.

28. The composition of claim 27, wherein the second silicone oil is selected from the group consisting of phenyl trimethicone, cetyl methicone, stearyl methicone, and combinations thereof.

29. The composition of claim 12, wherein the second silicone oil is present in an amount of from about 0.1% to about 30% by weight of the composition.

30. The composition of claim 1, further comprising a vitamin.

31. The composition of claim 30, wherein the vitamin is selected from the group consisting of vitamin E, vitamin K, vitamin C and derivatives thereof, vitamin A, B vitamins, and combinations thereof.

32. The composition of claim 30, wherein the vitamin is present in an amount of from about 0.001% to about 3% by weight of the composition.

33. The composition of claim 1, wherein the water added to the composition has a pH of from about 2 to about 9.

34. The composition of claim 1, wherein the composition is a spray product selected from the group consisting of perfume, air freshener, toner, moisturizer, acne treatment, sun screen, shine spray, hair colorant, and body spray.

35. A two-phase composition comprising:
(a) an aqueous phase comprising an alcohol;
(b) a silicone oil phase;
(c) guanine, wherein the guanine is present at the interface of (a) and (b); and, optionally, one or more of the following ingredients:
(i) pigment;
(ii) dye;
(iii) fragrance;
(iv) emollient;
(v) second silicone oil;
(vi) glycol;
(vii) vitamin;
(viii) sun screen;
(ix) sanitizing agent; and
(x) acne treatment agent.

36. A spray assembly comprising:
(a) a container comprising a transparent wall and a pump assembly extending from the wall;
(b) a liquid composition comprising a region containing pearlized oil droplets; and
(c) a fluorinated dip tube extending from the pump assembly into the region of pearlized oil droplets of the composition.

37. The spray assembly of claim 36, wherein the pump assembly is fluorinated.

38. The spray assembly of claim 36, wherein the wall is spherical.

39. The spray assembly of claim 36, wherein the wall is bulbous.

40. The spray assembly of claim 36, wherein the wall is conical.

41. The spray assembly of claim 36, wherein the transparent wall is formed at least in part of glass or a polymeric material.

42. The spray assembly of claim 41, wherein the transparent wall is glass.

43. The spray assembly of claim 41, wherein the polymeric material is selected from the group consisting of polyethylene terephthalate, polyvinylchloride, copolymers thereof, and combinations thereof.

44. The spray assembly of claim 36, wherein the dip tube, the pump assembly, or both are formed at least in part from a polymeric material selected from the group consisting of polyethylene polypropylene, co-polymers thereof, and blends thereof.

45. The spray assembly of claim 36, wherein the widest portion of the container has a radius of from about 1 cm to about 4 cm.

46. The spray assembly of claim 44, wherein the wall defines a height, and wherein half of the volume of the spray assembly is equal to or less than about half of the height.

47. The spray assembly of claim 36, wherein the pearlized oil droplets are present in an amount of from about 20% to about 50% by volume of the composition.

48. A spray assembly comprising:
(a) a container comprising a transparent wall and a pump assembly extending from the wall;
(b) the composition of claim 35; and
(c) a dip tube extending from the pump assembly into a region comprising the guanine-containing silicone oil phase oil.

49. The composition of claim 48, wherein the dip tube, the pump assembly, or both are formed at least in part from polyethylene that is fluorinated.
